(19) United States
(12) Patent Application Publication
(10) Pub. No.: US 2004/0180931 A1
(43) Pub. Date: Sep. 16, 2004

(54) Methods for treating Parkinson's disease

(76) Inventor: Raymond Pratt, Leonia, NJ (US)

Correspondence Address: Edward D. Grieff, HALE AND DORR LLP, 1455 Pennsylvania Avenue, NW, Washington, DC, 20004, US

(21) Appl. No.: 10/806,409

(22) Filed: Mar. 23, 2004

Related U.S. Application Data
(63) Continuation of application No. 10/321,653, filed on Dec. 18, 2002, now abandoned.
Application No. 10/321,653 is a continuation of application No. 09/899,028, filed on Jul. 6, 2001, now abandoned.
Application No. 09/899,028 is a continuation of application No. PCT/US01/07027, filed on Mar. 5, 2001.
(60) Provisional application No. 60/186,744, filed on Mar. 3, 2000.
(60) Provisional application No. 60/197,610, filed on Apr. 18, 2000.
(60) Provisional application No. 60/220,783, filed on Jul. 25, 2000.
(60) Provisional application No. 60/259,226, filed on Jan. 3, 2001.

(51) Int. Cl. A61K 31/445

(52) U.S. Cl. 514/319

(57) ABSTRACT

The invention provides methods for treating Parkinson's disease by administering a therapeutically effective amount of at least one cholinesterase inhibitor compound, a pharmaceutically acceptable salt thereof, or a stereoisomer thereof. A preferred cholinesterase inhibitor compound is donepezil, a pharmaceutically acceptable salt thereof, or a stereoisomer thereof.

### RELATED APPLICATIONS

This application is a continuation under § 120 of U.S. application Ser. No. 10/321,653 filed Dec. 18, 2002, which was a continuation under § 120 of U.S. application Ser. No. 09/899,028 filed Jul. 6, 2001, abandoned, which is a continuation under § 120 of PCT/US01/07027 filed Mar. 5, 2001, which claims priority under § 119 to U.S. Provisional Application No. 60/259,226 filed Jan. 3, 2001, U.S. Provisional Application No. 60/220,783 filed Jul. 25, 2000, U.S. Provisional Application No. 60/197,610 filed Apr. 18, 2000, and U.S. Provisional Application No. 60/186,744 filed Mar. 3, 2000.

### FIELD OF THE INVENTION

The invention provides safe and effective methods for treating Parkinson's disease by administering a therapeutically effective amount of at least one cholinesterase inhibitor compound to a patient. A preferred cholinesterase inhibitor compound is donepezil.

### BACKGROUND OF THE INVENTION

Parkinson's disease is a chronic nervous disease characterized by fine, slowly spreading tremors, rigidity, and a characteristic gait. Although the onset of Parkinson's disease may be abrupt, it generally occurs gradually. The initial symptom is often a fine tremor beginning in either a hand or a foot which may spread until it involves all of the members. The duration of Parkinson's disease is indefinite, and recovery rarely if ever occurs. Cognitive impairments and dementia may be seen in the later stages of Parkinson's disease, which is a common and significant source of morbidity.

Levodopa has historically been the medication of choice in treating Parkinson's disease, and there are rarely any failures with levodopa therapy in the early years of treatment. Unfortunately, this response is not sustainable. Most patients develop adverse effects after long-term usage of levodopa; in fact, in some the benefits of treatment wane as the disease progresses. Several common types of central nervous system dysfunction and peripheral side effects are associated with administration of levodopa. Toxic side effects to the central nervous system include mental changes, such as confusion, agitation, hallucinosis, hallucinations, delusions, depression, mania and excessive sleeping. The symptoms may be related to activation of dopamine receptors in non-striatal regions, particularly the cortical and limbic structures. All patients with Parkinson's disease, regardless of age, can develop cognitive impairments or dementia if they take excess amounts of levodopa as a means to overcome “off” periods. This is difficult to remedy, as reducing the dosage of levodopa may lessen its beneficial influence on motor function.

There is a need in the art for new and improved compounds for treating Parkinson's disease. The present invention is directed to this, as well as other, important ends.

### SUMMARY OF THE INVENTION

The present invention describes novel methods for treating and preventing Parkinson's disease by administering to a patient a therapeutically effective amount of at least one cholinesterase inhibitor compound. The methods for treating and preventing Parkinson's disease include methods for treating and preventing the motor dysfunctions, cognitive impairments, and/or dementia associated with Parkinson's disease. The cholinesterase inhibitor compound is preferably donepezil or a stereoisomer thereof.

These and other aspects of the invention are described in more detail below.

### BRIEF DESCRIPTION OF THE FIGURES

FIG. 1 is a graph showing the mean Mini-Mental State Examination (MMSE) score for patients receiving donepezil or placebo in weeks 1 through 10 and then alternately receiving placebo or donepezil in weeks 10 through 20. FIG. 1 shows that donepezil produced statistically significant treatment effects for the MMSE score (p=0.013). The MMSE score difference from baseline to week 10 was 2.1 points on donepezil and 0.3 points on placebo.

FIG. 2 is a graph showing the CIBIC+score for patients receiving donepezil or placebo in weeks 1 through 10 and then alternately receiving placebo or donepezil in weeks 10 through 20. FIG. 2 shows that donepezil produced statistically significant treatment effects for the CIBIC+score (p=0.034). The mean CIBIC+score at week 10 was 3.3 on donepezil and 4.1 on placebo.

FIG. 3 is a graph showing the CIBIC+response rates for patients receiving donepezil or placebo for 10 weeks. Improvement (i.e., a CIBIC+score of 3 or lower) was found for 5 (42%) patients on donepezil and 2 (17%) patients on placebo.

FIG. 4 is a graph showing the severity of parkinsonism for patients receiving donepezil or placebo for 10 weeks. The baseline (i.e., week 0) Unified Parkinson's Disease Rating Scale (UPDRS) is shown at the far left in FIG. 4. The scorings on the UPDRS motor subscale disclosed no deterioration in parkinsonian symptoms on donepezil at week 10 (p=0.37). The caregivers and patients did not report subjective worsening of parkinsonism on donepezil.

### DETAILED DESCRIPTION OF THE INVENTION

“Parkinson's disease” is a neurological syndrome usually resulting from deficiency of the neurotransmitter dopamine as the consequence of degenerative, vascular or inflammatory changes in the basal ganglia, and is characterized by motor dysfunctions, cognitive dysfunctions and/or dementia.

“Motor dysfunctions” include one or more of muscle tremors, rigidity, a characteristic gait, droopy posture, and/or masklike facies. The “motor dysfunctions” of Parkinson's disease may also be referred to as parkinsonism.

“Cognitive impairment” refers to an acquired deficit in one or more of memory function, problem solving, orientation and/or abstraction that impinges on a patient's ability to function independently.

“Dementia” refers to a global deterioration of intellectual functioning in clear consciousness, and is characterized by one or more symptoms of disorientation, impaired memory, impaired judgment, and/or impaired intellect. The symptoms of “dementia” are generally worse than, and may encompass, the symptoms of cognitive impairment.

“Patient” refers to animals, preferably mammals, more preferably humans. The term “patient” includes men and women.

In each of the methods described herein, the cholinesterase inhibitors of the invention alleviate (e.g., do not change, reduce, or eliminate) at least one symptom of Parkinson's disease. In some embodiments, a successful treatment may be a treatment in which the symptoms of Parkinson's disease do not change, i.e., the symptoms neither improve nor worsen. In other embodiments, a successful treatment may be a treatment in which the symptoms of Parkinson's disease are reduced or eliminated. The symptoms may be motor dysfunctions, cognitive dysfunctions, and/or dementia.

In the methods for treating Parkinson's disease a patient is administered a therapeutically effective amount of at least one cholinesterase inhibitor of formula I or a pharmaceutically acceptable salt thereof:

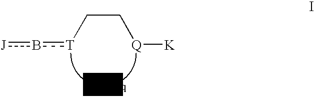

wherein J is

(a) a substituted or unsubstituted group selected from the group consisting of (1) phenyl, (2) pyridyl, (3) pyrazyl, (4) quinolyl, (5) cyclohexyl, (6) quinoxalyl, and (7) furyl;

(b) a monovalent or divalent group, in which the phenyl may have one or more substituents selected from (1) indanyl, (2) indanonyl, (3) indenyl, (4) indenonyl, (5) indanedionyl, (6) tetralonyl, (7) benzosuberonyl, (8) indanolyl, and (9) C₆H₅—CO—CH(CH₃)—;

(c) a monovalent group derived from a cyclic amide compound;

(d) a lower alkyl group; or

(e) a group of R²¹—CH═CH—, in which R²¹ is hydrogen or a lower alkoxycarbonyl group;

B is —(CHR²²)ᵣ—, —CO—(CHR²²)ᵣ—, —NR⁴—(CHR²²)ᵣ—, —CO—NR⁵—(CHR²²)ᵣ—, —CH═CH—(CHR²²)ᵣ—, —OCOO—(CHR²²)ᵣ—, —OOC—NH—(CHR²²)ᵣ—, —NH—CO—(CHR²²)ᵣ—, —CH₂—CO—NH—(CHR²²)ᵣ—, —(CH₂)₂—NH—(CHR²²)ᵣ—, —CH(OH)—(CHR²²)ᵣ—, ═(CH—CH═CH)_{b}—, ═CH—(CH₂)_{c}—, ═(CH—CH)_{d}═, —CO—CH═CH—CH₂—, —CO—CH₂—CH(OH)—CH₂—, —CH(CH₃)—CO—NH—CH₂—, —CH═CH═CO—NH—(CH₂)₂—, —NH—, —O—, —S—, a dialkylaminoalkyl-carbonyl or a lower alkoxycarbony;

wherein R⁴ is hydrogen, lower alkyl, acyl, lower alkylsulfonyl, phenyl, substituted phenyl, benzyl, or substituted benzyl; R⁵is hydrogen, lower alkyl or phenyl; r is zero or an integer of about 1 to about 10; R²² is hydrogen or methyl so that one alkylene group may have no methyl branch or one or more methyl branches; b is an integer of about 1 to about 3; c is zero or an integer of about 1 to about 9; d is zero or an integer of about 1 to about 5;

T is nitrogen or carbon;

Q is nitrogen, carbon or

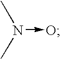

q is an integer of about 1 to about 3;

K is hydrogen, phenyl, substituted phenyl, arylalkyl in which the phenyl may have a substituent, cinnamyl, a lower alkyl, pyridylmethyl, cycloalkylalkyl, adamantanemethyl, furylmenthyl, cycloalkyl, lower alkoxycarbonyl or an acyl; and

[symbol] is a single bond or a double bond.

In the compound of formula I, J is preferably (a) or (b), more preferably (b). In the definition of (b), a monovalent group (2), (3) and (5) and a divalent group (2) are preferred. The group (b) preferably includes, for example, the groups having the formulae shown below:

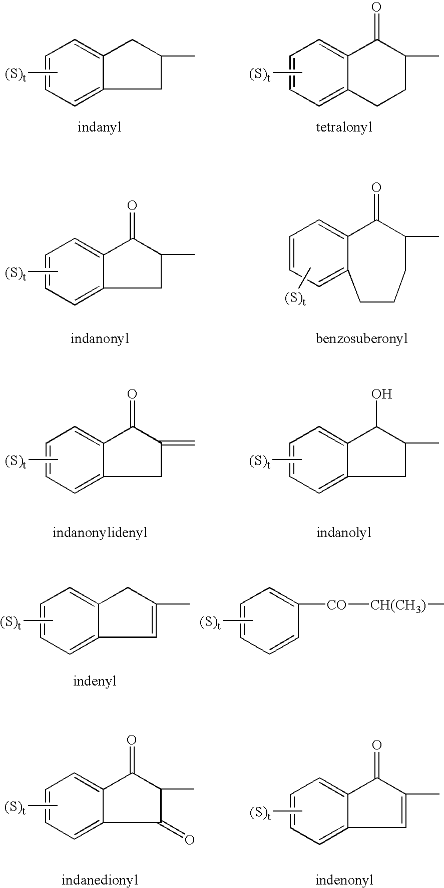

wherein t is an integer of about 1 to about 4; and each S is independently hydrogen or a substituent, such as a lower alkyl having 1 to 6 carbon atoms or a lower alkoxy having 1 to 6 carbon atoms. Among the substituents, methoxy is most preferred. The phenyl is most preferred to have 1 to 3 methoxy groups thereon. (S)ₜ may form methylene dioxy groups or ethylene dioxy groups on two adjacent carbon atoms of the phenyl group. Of the above groups, indanonyl, indanedionyl and indenyl, optionally having substituents on the phenyl, are the most preferred.

In the definition of B, —(CHR²²)ᵣ—, —CO—(CHR²²)ᵣ—, ═(CH—CH═CH)_{b}—, ═CH—(CH₂)_{c}— and ═(CH—CH)_{d}═ are preferable. The group of —(CHR²²)ᵣ— in which R²² is hydrogen and r is an integer of 1 to 3, and the group of ═CH—(CH₂)_{c}— are most preferable. The preferable groups of B can be connected with (b) of J, in particular (b)(2).

The ring containing T and Q in formula I can be 5-, 6- or 7-membered. It is preferred that Q is nitrogen, T is carbon or nitrogen, and q is 2; or that Q is nitrogen, T is carbon, and q is 1 or 3; or that Q is carbon, T is nitrogen and q is 2.

It is preferable that K is a phenyl, arylalkyl, cinnamyl, phenylalkyl or a phenylalkyl having a substituent(s) on the phenyl.

In preferred embodiments, the cyclic amine compounds of formula I are the piperidine compounds of formula II or a pharmaceutically acceptable salt thereof:

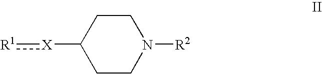

wherein R¹ is a (1) substituted or unsubstituted phenyl group; (2) a substituted or unsubstituted pyridyl group; (3) a substituted or unsubstituted pyrazyl group; (4) a substituted or unsubstituted quinolyl group; (5) a substituted or unsubstituted indanyl group; (6) a substituted or unsubstituted cyclohexyl group; (7) a substituted or unsubstituted quinoxalyl group; (8) a substituted or unsubstituted furyl group; (9) a monovalent or divalent group derived from an indanone having a substituted or unsubstituted phenyl ring; (10) a monovalent group derived from a cyclic amide compound; (11) a lower alkyl group; or (12) a group of the formula R³—CH═C—, where R³ is a hydrogen atom or a lower alkoxycarbonyl group;

X is —(CH₂)ₙ—, —C(O)—(CH₂)ₙ—, —N(R⁴)—(CH₂)ₙ—, —C(O)—H(R⁵)—(CH₂)ₙ—, —CH═CH—(CH₂)ₙ—, —O—C(O)—O—(CH₂)ₙ—, —O—C(O)—NH—(CH₂)ₙ—, —CH═CH—CH═CO—, —NH—C(O)—(CH₂)ₙ—, —CH₂—C(O)—NH—(CH₂)ₙ—, —(CH₂)₂—C(O)—NH—(CH₂)ₙ—, —CH(OH)—(CH₂)ₙ—, —C(O)—CH═CH—CH₂—, —C(O)—CH₂—CH(OH)—CH₂—, —CH(CH₃)—C(O)—NH—CH₂—, —CH═CH—C(O)—NH—(CH₂)₂—, a dialkylaminoalkylcarbonyl group, a lower alkoxycarbonyl group;

where n is an integer of 0 to 6; R⁴ is a hydrogen atom, a lower alkyl group, an acyl group, a lower alkylsulfonyl group, a substituted or unsubstituted phenyl group, or a substituted or unsubstituted benzyl group; and R⁵ is a hydrogen atom a lower alkyl group or a phenyl group;

R² is a substituted or unsubstituted phenyl group; a substituted or unsubstituted arylalkyl group; a cinnamyl group; a lower alkyl group; a pyridylmethyl group; a cycloalkylalkyl group; an adamantanemethyl group; or a furoylmethyl group; and

[symbol] is a single bond or a double bond.

The term “lower alkyl group” as used herein means a straight or branched alkyl group having 1 to 6 carbon atoms. Exemplary “lower alkyl groups” include methyl, ethyl, propyl, isopropyl, butyl, isobutyl, sec-butyl, tert-butyl, pentyl (amyl), isopentyl, neopentyl, tert-pentyl, 1-methylbutyl, 2-methylbutyl, 1,2-dimethylpropyl, hexyl, isohexyl, 1-methylpentyl, 2-methyl-pentyl, 3-methylpentyl, 1,1-dimethylbutyl, 1,2-dimethylbutyl, 2,2-dimethylbutyl, 1,3-dimthyl-butyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, and the like. The lower alkyl group is preferably methyl, ethyl, propyl or isopropyl; more preferably methyl.

Specific examples of the substituents for the substituted or unsubstituted phenyl, pyridyl, pyrazyl, quinolyl, indanyl, cyclohexyl, quinoxalyl and furyl groups in the definition of R¹ include lower alkyl groups having 1 to 6 carbon atoms, such as methyl, ethyl, n-propyl, isopropyl, n-butyl, isobutyl, and tert-butyl groups; lower alkoxy groups corresponding to the above-described lower alkyl groups, such as methoxy and ethoxy groups; a nitro group; halogen atoms, such as chlorine, fluorine and bromine; a carboxyl group; lower alkoxycarbonyl groups corresponding to the above-described lower alkoxy groups, such as methoxycarbonyl, ethoxycarbonyl, isopropoxycarbonyl, n-propoxycarbonyl, and n-butyloxycarbonyl groups; an amino group; a lower monoalkylamino group; a lower dialkylamino group; a carbamoyl group; acylamino groups derived from aliphatic saturated monocarboxylic acids having 1 to 6 carbon atoms, such as acetylamino, propionylamino, butyrylamino, isobutyrylamino, valerylamino, and pivaloylamino groups; cycloalkyloxycarbonyl groups, such as a cyclohexyloxycarbonyl group; lower alkylaminocarbonyl groups, such as methylaminocarbonyl and ethylaminocarbonyl groups; lower alkylcarbonyloxy groups corresponding to the above-defined lower alkyl groups, such as methylcarbonyloxy, ethylcarbonyloxy, and n-propylcarbonyloxy groups; halogenated lower alkyl groups, such as a trifluoromethyl group; a hydroxyl group; a formyl group; and lower alkoxy lower alkyl groups, such as ethoxymethyl, methoxymethyl and methoxyethyl groups. The “lower alkyl groups” and “lower alkoxyl groups” in the above description of the substituent include all the groups derived from the above-mentioned groups. The substituent may be one to three of them, which may be the same or different.

When the substituent is a phenyl group, the following group is within the scope of the substituted phenyl group:

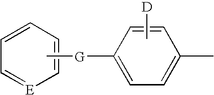

wherein G is —C(O)—, —O—C(O)—, —O—, —CH₂—NH—C(O)—, —CH₂—O—, —CH₂—SO₂—, —CH(OH)—, or —CH₂—S(→O)—; E is a carbon or nitrogen atom; and D is a substituent.

Preferred examples of the substituents (i.e., “D”) for the phenyl group include lower alkyl, lower alkoxy, nitro, halogenated lower alkyl, lower alkoxycarbonyl, formyl, hydroxyl, and lower alkoxy lower alkyl groups, halogen atoms, and benzyol and benzylsulfonyl groups. The substituent may be two or more of them, which may be the same or different.

Preferred examples of the substituent for the pyridyl group include lower alkyl and amino groups and halogen atoms.

Preferred examples of the substituent for the pyrazyl group include lower alkoxycarbonyl, carboxyl, acylamino, carbamoyl, and cycloalkyloxycarbonyl groups.

With respect to R¹, the pyridyl group is preferably a 2-pyridyl, 3-pyridyl, or 4-pyridyl group; the pyrazyl group is preferably a 2-pyrazinyl group; the quinolyl group is preferably a 2-quinolyl or 3-quinolyl group; the quinoxalinyl group is preferably a 2-quinoxalinyl or 3-quinoxalinyl group; and the furyl group is preferably a 2-furyl group.

Specific examples of preferred monovalent or divalent groups derived from an indanone having an unsubstituted or substituted phenyl ring include those represented by formulas (A) and (B):

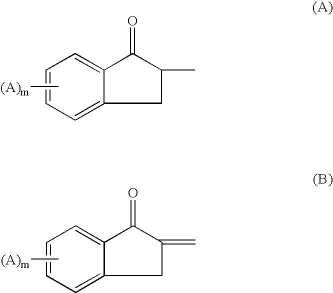

where m is an integer of from 1 to 4, and each A is independently a hydrogen atom, a lower alkyl group, a lower alkoxy group, a nitro group, a halogen atom, a carboxyl group, a lower alkoxycarbonyl group, an amino group, a lower monoalkylamino group, a lower dialkylamino group, a carbamoyl group, an acylamino group derived from aliphatic saturated monocarboxylic acids having 1 to 6 carbon atoms, a cycloalkyloxycarbonyl group, a lower alkylaminocarbonyl group, a lower alkylcarbonyloxy group, a halogenated lower alkyl group, a hydroxyl group, a formyl group, or a lower alkoxy lower alkyl group; preferably a hydrogen atom, a lower alkyl group or a lower alkoxy group; most preferably the indanone group is unsubstituted or substituted with 1 to 3 methoxy groups.

Examples of the monovalent group derived from a cyclic amide compound include quinazolone, tetrahydroisoquinolinone, tetrahydrobenzodiazepinone, and hexahydrobenzazocinone. However, the monovalent group may be any one having a cyclic amide group in the structural formula thereof, and is not limited to the above-described specific examples. The cyclic amide group may be one derived from a monocyclic or condensed heterocyclic ring. The condensed heterocyclic ring is preferably one formed by condensation with a phenyl ring. In this case, the phenyl ring may be substituted with a lower alkyl group having 1 to 6 carbon atoms, preferably a methyl group, or a lower alkoxy group having 1 to 6 carbon atoms, preferably a methoxy group.

Preferred examples of the monovalent group include the following:

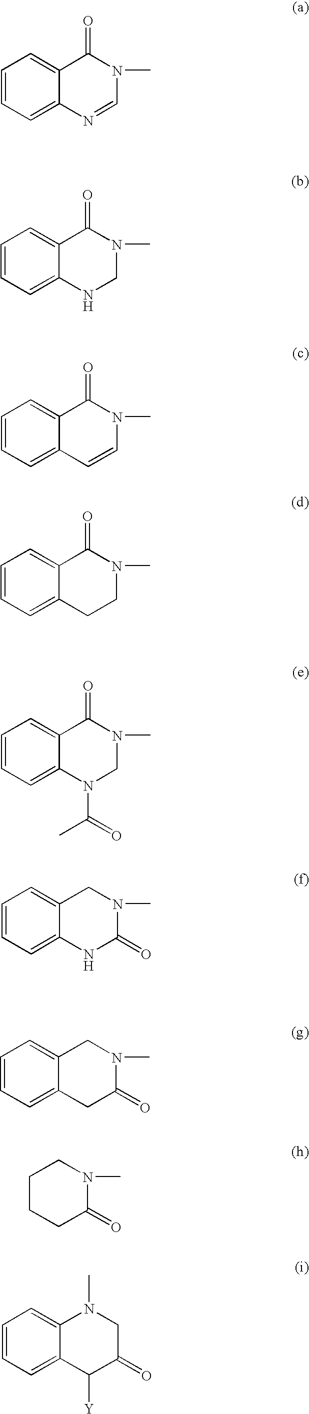

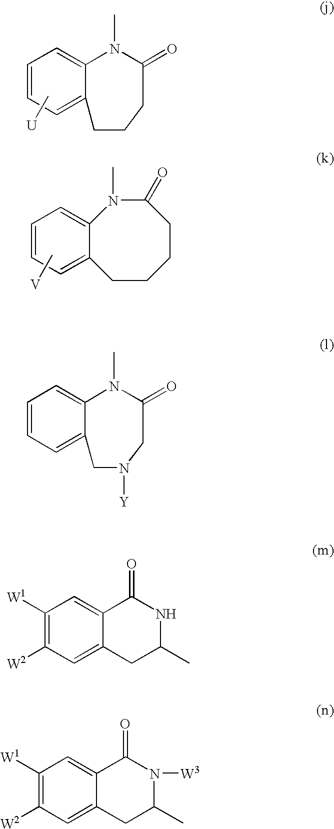

In the above formulae, Y is a hydrogen atom or a lower alkyl group; V and U are each a hydrogen atom or a lower alkoxy group (preferably dimethoxy); W¹and W² are each a hydrogen group. The right hand ring in formulae (j) and (l) is a 7-membered ring, while the right hand ring in formula (k) is an 8-membered ring.

The most preferred examples of the above-defined R¹ include a monovalent group derived from an indanone having an unsubstituted or substituted phenyl group and a monovalent group derived from a cyclic amide compound.

The most preferred examples of the above-defined X include —(CH₂)ₙ—, an amide group, or groups represented by the above formulae where n is 2. Thus, it is most preferred that any portion of a group represented by the formula R¹ [symbol] X [symbol] have a carbonyl or amide group.

The substituents involved in the expressions “a substituted or unsubstituted phenyl group” and “a substituted or unsubstituted arylalkyl group” in the above definition of R² are the same substituents as those described for the above definitions of a phenyl group, a pyridyl group, a pyrazyl group, a quinolyl group, an indanyl group, a cyclohexyl group, a quinoxalyl group or a furyl group in the definition of R¹.

The term “arylalkyl group” is intended to mean an unsubstituted benzyl or phenethyl group or the like.

Specific examples of the pyridylmethyl group include 2-pyridylmethyl, 3-pyridylmethyl, and 4-pyridylmethyl groups.

Preferred examples of R² include benzyl and phenethyl groups. The symbol [symbol] means a double or single bond. The bond is a double bond only when R¹ is the divalent group (B) derived from an indanone having an unsubstituted or substituted phenyl ring, while it is a single bond in other cases.

In preferred embodiments, the compound of formula II is a compound of formula III or a pharmaceutically acceptable salt thereof:

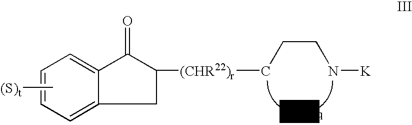

wherein r is an integer of about 1 to about 10; each R²² is independently hydrogen or methyl; K is a phenalkyl or a phenalkyl having a substituent on the phenyl ring; each S is independently a hydrogen, a lower alkyl group having 1 to 6 carbon atoms or a lower alkoxy group having 1 to 6 carbon atoms; t is an integer of 1 to 4; q is an integer of about 1 to about 3; with the proviso that (S)ₜ can be a methylenedioxy group or an ethylenedioxy group joined to two adjacent carbon atoms of the phenyl ring.

In preferred embodiments, the compound of formula III is: 1-benzyl-4-((5,6-dimethoxy-1-indanon)-2-yl)methylpiperidine; 1-benzyl-4-((5,6-dimethoxy-1-indanon)-2-ylidenyl)methylpiperidine; 1-benzyl-4-((5-methoxy-1-indanon)-2-yl)methylpiperidine; 1-benzyl-4-((5,6-diethoxy- 1-indanon)-2-yl)methylpiperidine; 1-benzyl-4-((5,6-methnylenedioxy-1-indanon)-2-yl)methylpiperidine; 1-(m-nitrobenzyl)-4-((5,6-dimethoxy-1-indanon)-2-yl)methylpiperidine; 1-cyclohexylmethyl-4-((5,6-dimethoxy-1-indanon)-2-yl)methylpiperidine; 1-(m-fluorobenzyl)-4-((5,6-dimethoxy-1-indanon)-2-yl)methylpiperidine; 1-benzyl-4-((5,6-dimethoxy- 1-indanon)-2-yl)propylpiperidine; 1-benzyl-4-((5-isopropoxy-6-methoxy-1-indanon)-2-yl)methylpiperidine; 1-benzyl-4-((5,6-dimethoxy-1-oxoindanon)-2-yl)propenylpiperidine; or pharmaceutically acceptable salts thereof.

In more preferred embodiments, the compound of formula III is 1-benzyl-4-((5,6-dimethoxy-1-indanon)-2-yl)methylpiperidine or a pharmaceutically acceptable salt thereof, which is also known as donepezil, and which has formula IV:

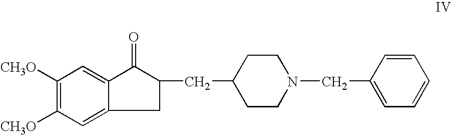

or a stereoisomer thereof.

In the most preferred embodiment, the compound of formula IV is 1-benzyl-4-((5,6-dimethoxy-1-indanon)-2-yl)methylpiperidine hydrochloride, which is also known as donepezil hydrochloride, and which has formula V:

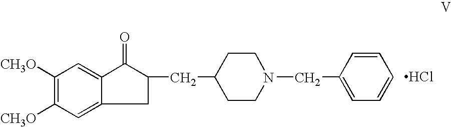

or a stereoisomer thereof.

The compounds of the present invention may have an asymmetric carbon atom(s), depending upon the substituents, and can have stereoisomers, which are within the scope of the invention. For example, donepezil hydrochloride can be in the forms described in Japanese Patent Application Nos. 4-187674 and 4-21670, the disclosures of which are incorporated by reference herein in their entirety. Japanese Patent Application No. 4-187674 describes a compound having the formula VI:

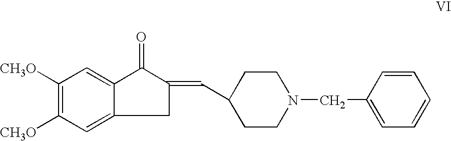

which can be in the form of a pharmaceutically acceptable salt, such as a hydrochloride salt. Japanese Patent Application No. 4-21670 describes compounds having the formula VII:

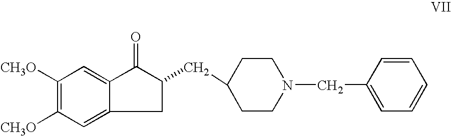

which can be in the form of a pharmaceutically acceptable salt, such as a hydrochloride salt; and compounds of the formula VIII:

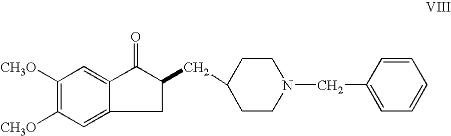

which can be in the form of a pharmaceutically acceptable salt, such as a hydrochloride salt; and compounds of the formula IX:

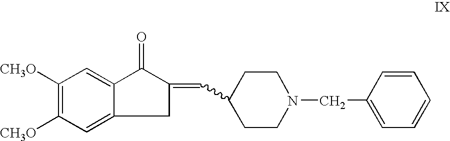

which can be in the form of a pharmaceutically acceptable salt, such as a hydrochloride salt.

The compounds of the invention may be administered in the form of a pharmaceutically acceptable salt. Pharmaceutically acceptable salts are known in the art and include those of inorganic acids, such as hydrochloride, sulfate, hydrobromide and phosphate; and those of organic acids, such as formate, acetate, trifluoroacetate, methanesulfonate, benzenesulfonate and toluenesulfonate. When certain substituents are selected, the compounds of the present invention may form, for example, alkali metal salts, such as sodium or potassium salts; alkaline earth metal salts, such as calcium or magnesium salts; organic amine salts, such as a salt with trimethylamine, triethylamine, pyridine, picoline, dicyclohexylamine or N,N′-dibenzylethylene-diamine. One skilled in the art will recognize that the compounds of the invention may be made in the form of any other pharmaceutically acceptable salt.

The compounds of the invention may be prepared by processes that are known in the art and described, for example, in U.S. Pat. No. 4,895,841, WO 98/39000, and Japanese Patent Application Nos. 4-187674 and 4-21670, the disclosures of each of which are incorporated by reference herein in their entirety. Donepezil hydrochloride, a preferred cholinesterase inhibitor for use in the methods described herein, is commercially available as ARICEPT® from Eisai Inc., Teaneck, N.J.

The dosage regimen for treating Parkinson's disease with the cholinesterase inhibitors described herein is selected in accordance with a variety of factors, including the age, weight, and medical condition of the patient, the severity of the disease, the route of administration, pharmacological considerations such as the activity, efficacy, pharmacokinetic and toxicology profiles of the particular cholinesterase inhibitor used, whether a drug delivery system is used and whether the cholinesterase inhibitor is administered as part of a drug combination.

In preferred embodiments, the cholinesterase inhibitors of the invention are administered in doses of about 0.1 milligram to about 300 milligrams per day, preferably about 1 milligram to about 100 milligrams per day, more preferably about 5 milligrams to about 10 milligrams per day. The doses can be administered in one to four portions over the course of a day, preferably once a day.

In preferred embodiments of the methods described herein, a physician may administer patients donepezil hydrochloride, which is commercially available as ARICEPT® (Eisai Inc., Teaneck, N.J.). Donepezil hydrochloride is commercially available in amounts of 5 milligrams and 10 milligrams. The tablets may be administered one to about four times a day. In preferred embodiments, one 5 milligram or one 10 milligram ARICEPT® tablet is administered once a day for the methods described herein.

The cholinesterase inhibitors of the invention may be administered orally, topically, parenterally, by inhalation (nasal or oral), or rectally in dosage unit formulations containing conventional nontoxic pharmaceutically acceptable carriers, adjuvants, and vehicles as desired. The term parenteral as used herein includes subcutaneous, intravenous, intramuscular, intrasternal injection, or infusion techniques. Preferably, the cholinesterase inhibitors of the invention are orally administered, preferably as tablets.

Injectable preparations, for example, sterile injectable aqueous or oleaginous suspensions may be formulated according to the known art using suitable dispersing or wetting agents, suspending agents (e.g., methylcellulose, Polysorbate 80, hydroxyethylcellulose, acacia, powdered tragacanth, sodium carboxymethylcellulose, polyoxytehylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like) and preservatives. The sterile injectable preparation may also be a sterile injectable solution or suspension in a nontoxic parenterally acceptable diluent or solvent, for example, as a solution in 1,3-butanediol. Among the acceptable vehicles and solvents that may be used are water, Ringer's solution, and isotonic sodium chloride solution. In addition, sterile, fixed oils are conventionally used as a solvent or suspending medium. For this purpose any bland fixed oil may be used including synthetic mono- or diglycerides, in addition, fatty acids such as oleic acid find use in the preparation of injectables. The preparations can be lyophilized by methods known in the art.

Solid dosage forms for oral administration may include chewing gum, capsules, tablets, sublingual tablets, powders, granules and gels; most preferably tablets. In such solid dosage forms, the active compound may be admixed with one or more inert diluents such as lactose or starch. As is normal practice, such dosage forms may also comprise other substances including lubricating agents such as magnesium stearate. In the case of capsules, tablets, and pills, the dosage forms may also comprise buffering agents. The tablets can be prepared with enteric or film coatings, preferably film coatings.

In addition to the active ingredient, the tablets may comprise lactose monohydrate, corn starch, microcrystalline cellulose, hydroxypropyl cellulose, and magnesium stearate; while the film-coating on the tablet may comprises talc, polyethylene glycol, hydroxpropyl methylcellulose, titanium dioxide, and, optionally, other coloring agents, such as yellow iron oxide.

Liquid dosage forms for oral administration can include pharmaceutically acceptable emulsions, solutions, suspensions, and syrups containing inert diluents commonly used in the art, such as water. Such compositions can also comprise adjuvants, such as wetting agents, emulsifying and suspending agents, and sweetening, flavoring, and perfuming agents.

For administration by inhalation, the compositions of the invention can be delivered from an insufflator, a nebulizer or a pressured pack or other convenient mode of delivering an aerosol spray. Pressurized packs can include a suitable propellant. Alternatively, for administration by inhalation, the compositions can be administered in the form of a dry powder composition or in the form of a liquid spray.

Suppositories for rectal administration can be prepared by mixing the active compounds with suitable nonirritating excipients such as cocoa butter and polyethylene glycols that are solid at room temperature and liquid at body temperature.

For topical administration to the epidermis, the cholinesterase inhibitors of the invention can be formulated as ointments, creams or lotions, or as the active ingredient of a transdermal patch. Ointments and creams may, for example, be formulated with an aqueous or oily base with the addition of suitable thickening and/or gelling agents. Lotions may be formulated with an aqueous or oily base and can also generally contain one or more emulsifying agents, stabilizing agents, dispersing agents, suspending agents, thickening agents, and/or coloring agents. The cholinesterase inhibitors can also be administered via iontophoresis.

While the cholinesterase inhibitors of the invention may be administered as the sole active pharmaceutical agent in the methods described herein, they can also be used in combination with one or more compounds which are known to be therapeutically effective against Parkinson's disease.

### EXAMPLES

The following example is for purposes of illustration and is not intended to limit the scope of the appended claims.

### Example 1

This example was conducted to study the safety and efficacy of donepezil in patients with Parkinson's disease who were suffering from dementia. Cognitive impairments may occur in 70 to 80% of patients with Parkinson's disease, and there is a six-fold risk for dementia in patients with Parkinson's disease compared to elderly subjects without Parkinson's disease. Aarsland et al, Neurology, 56(Suppl. 3):A128, Abstract No. P02.105 (April 2001).

A randomized, placebo-controlled, double-blind crossover study was conducted with fourteen patients with Parkinson's disease (13 men, 1 woman; mean age 78 (SD 3.6) years; mean duration of illness 11.4 (SD 4.9) years) who had developed cognitive impairments (mean Mini-Mental State Examination (MMSE) score 20.7 (SD 3.3)) 8.6 (SD 5.8) years after the onset of parkinsonism. The patients were given 5 mg donepezil, 10 mg donepezil, or placebo during two sequential randomized periods lasting 10 weeks each. At weeks 6 and 10 the patients completed a battery of cognitive tests. Cognitive impairments were measured using the MMSE and the CIBIC, a 7-point scale of the clinician's interview-based impression of change based upon information scored independently of the MMSE. Parkinsonism was assessed using the Unified Parkinson's Disease Rating Scale (UPDRS).

The two groups were comparable at baseline. Two patients dropped out of the study after 1 and 4 weeks of the first treatment sequence while receiving donepezil due to adverse events. The remaining patients completed the study with few and minor side-effects. All efficacy measures showed a numerical benefit for donepezil. Analysis of variance considering the repeated measurements in the study design revealed no carry-over or learning effects.

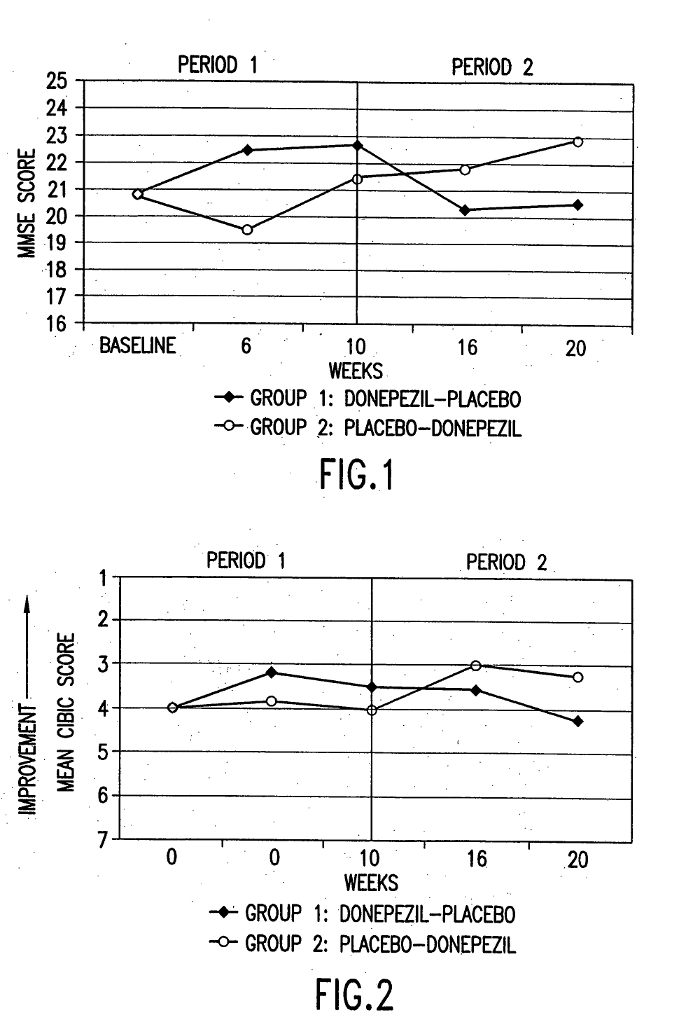

Statistically significant treatment effects were found with donepezil for the MMSE score, as shown in FIG. 1. The mean score of the MMSE, which served as the primary outcome, increased by 2.1 points (95% confidence interval (CI) 0.4-3.8) in the treatment group from start of treatment to week 10. On placebo, the mean change on the MMSE was 0.3 points (95% CI 1.8-2.8). A significant effect of donepezil compared to placebo was found (p=0.02).

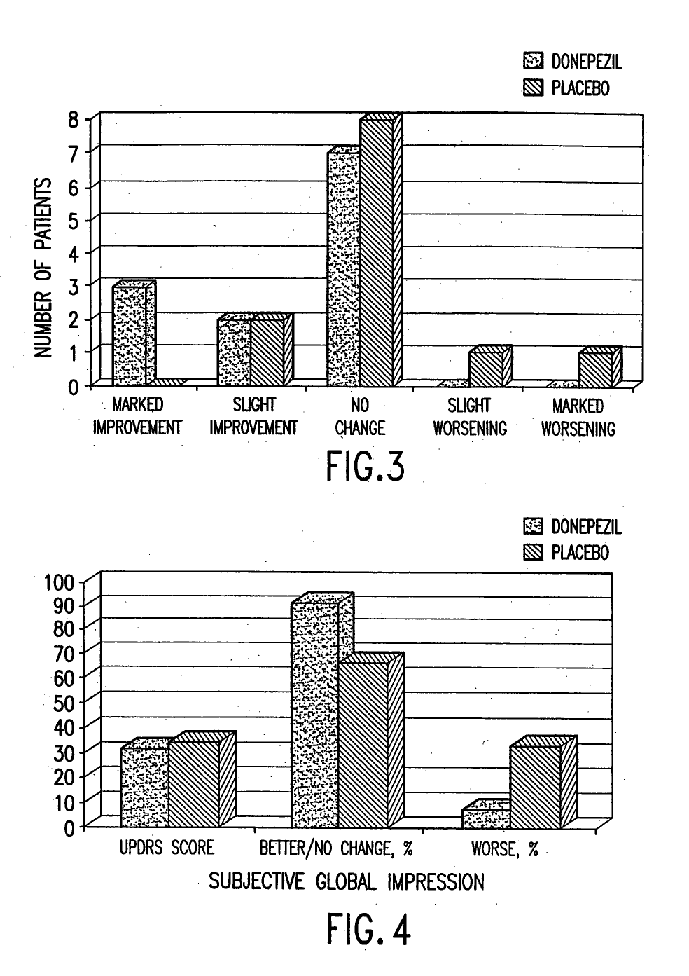

Statistically significant treatment effects were found for donepezil for the CIBIC+ score, as shown in FIG. 2. The mean CBIC score at week 10 was 3.3 on donepezil and was 4.1 on placebo. As shown in FIG. 3, improvement (i.e., a CIBIC score of 3 or lower) was found for 5 (42%) patients on donepezil and for 2 (17%) patients on placebo.

FIG. 4 shows that there was no deterioration in parkinsonism symptoms in the patients on donepezil at week 10 in the study.

In summary, patients with Parkinson's disease and mild or moderate dementia treated for 10 weeks with donepezil showed improvement in cognitive function. Donepezil was well tolerated, and parkinsonism did not worsen during treatment.

Each of the patents and publications cited herein are incorporated by reference herein in their entirety.

It will be apparent to one skilled in the art that various modifications can be made to the invention without departing from the spirit or scope of the appended claims.

What is claimed is:

1. A method for treating dementia in a patient with Parkinson's disease comprising orally administering about 5 milligrams to about 10 milligrams of a compound of formula IV or a pharmaceutically acceptable salt thereof, wherein the compound of formula IV is: 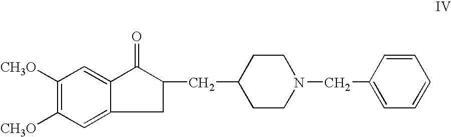
or a stereoisomer thereof.

2. The method of claim 1, wherein the pharmaceutically acceptable salt of the compound of formula IV is a hydrochloride salt.

3. The method of claim 1, wherein the stereoisomer of the compound of formula IV is a compound of formula VII or a pharmaceutically acceptable salt thereof, wherein the compound of formula VII is:

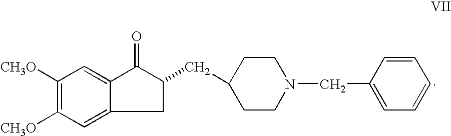

4. The method of claim 1, wherein the stereoisomer of the compound of formula IV is a compound of formula VIII or a pharmaceutically acceptable salt thereof, wherein the compound of formula VIII is:

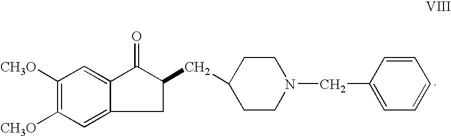

5. The method of claim 1, wherein the compound of formula IV, the pharmaceutically acceptable salt thereof, or the stereoisomer thereof is in the form of a tablet.

6. A method for treating dementia in a patient with Parkinson's disease comprising administering a therapeutically effective amount of a compound of formula IV or a pharmaceutically acceptable salt thereof,
wherein the compound of formula IV is: 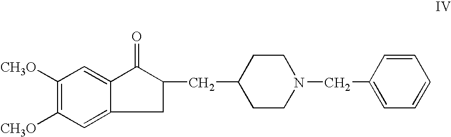
or a stereoisomer thereof.

7. The method of claim 6, wherein the pharmaceutically acceptable salt of the compound of formula IV is a hydrochloride salt.

8. The method of claim 6, wherein the stereoisomer of the compound of formula IV is a compound of formula VII or a pharmaceutically acceptable salt thereof, wherein the compound of formula VII is:

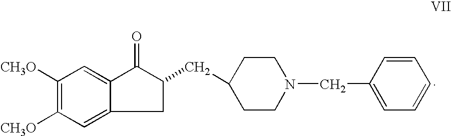

9. The method of claim 6, wherein the stereoisomer of the compound of formula IV is a compound of formula VIII or a pharmaceutically acceptable salt thereof, wherein the compound of formula VIII is:

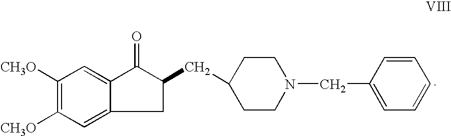

10. The method of claim 6, wherein the compound of formula IV, the pharmaceutically acceptable salt thereof, or the stereoisomer thereof is administered in an amount of about 1 milligram to about 100 milligrams.

11. The method of claim 6, wherein the compound of formula IV, the pharmaceutically acceptable salt thereof, or the stereoisomer thereof is transdermally administered to the patient.

12. The method of claim 11, wherein the compound of formula IV, the pharmaceutically acceptable salt thereof, or the stereoisomer thereof is transdermally administered to the patient in the form of a transdermal patch.

13. The method of claim 6, wherein the compound of formula IV, the pharmaceutically acceptable salt thereof, or the stereoisomer thereof is orally administered to the patient.

14. The method of claim 13, wherein the compound of formula IV, the pharmaceutically acceptable salt thereof, or the stereoisomer thereof is orally administered to the patient in the form of a tablet.

15. A method for treating Parkinson's disease in a patient in need thereof comprising administering a therapeutically effective amount of a compound of formula IV or a pharmaceutically acceptable salt thereof, wherein the compound of formula IV is: 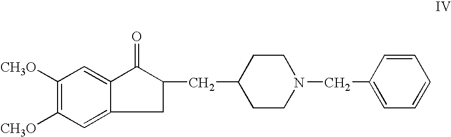
or a stereoisomer thereof.

16. The method of claim 15, wherein the pharmaceutically acceptable salt of the compound of formula IV is a hydrochloride salt.

17. The method of claim 15, wherein the stereoisomer of the compound of formula IV is a compound of formula VII or a pharmaceutically acceptable salt thereof, wherein the compound of formula VII is:

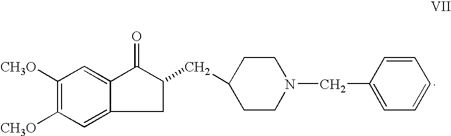

18. The method of claim 15, wherein the stereoisomer of the compound of formula IV is a compound of formula VIII or a pharmaceutically acceptable salt thereof, wherein the compound of formula VIII is:

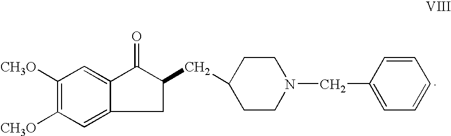

19. The method of claim 15, wherein the compound of formula IV, the pharmaceutically acceptable salt thereof, or the stereoisomer thereof is administered in an amount of about 1 milligrams to about 100 milligrams.

20. The method of claim 15, wherein the compound of formula IV, the pharmaceutically acceptable salt thereof, or the stereoisomer thereof is administered in an amount of about 5 milligrams or about 10 milligrams.
